(19) United States
(12) Patent Application Publication
(10) Pub. No.: US 2024/0100249 A1
(43) Pub. Date: Mar. 28, 2024

(54) METHOD FOR CONTROLLING ELECTROCHEMICAL PUMP AND ELECTROCHEMICAL PUMP IMPLEMENTING THE SAME

(71) Applicant: MICROMED CO., LTD., Taipei (TW)

(72) Inventors: Po-Ying LI, New Taipei City (TW); Tsung-Chieh CHENG, New Taipei City (TW); Jiunn-Ru LAI, Kaohsiung City (TW)

(21) Appl. No.: 18/531,346

(22) Filed: Dec. 6, 2023

Related U.S. Application Data
(63) Continuation-in-part of application No. 17/178,900, filed on Feb. 18, 2021.
(60) Provisional application No. 62/979,772, filed on Feb. 21, 2020.

(51) Int. Cl. A61M 5/145; A61M 5/172

(52) U.S. Cl. CPC A61M 5/14526; A61M 5/172; A61M 2205/0277

(57) ABSTRACT

Disclosed is a method for controlling an electrochemical pump, comprising: providing an electrochemical pump, and causing a control circuit of the electrochemical pump to generate a pulse signal to enable an electrochemical reaction on electrodes of the electrochemical pump, wherein the pulse signal has alternating on-periods and off-periods where the pulse signal is off, and wherein each on-period has a plurality of on-times where the pulse signal is on and a plurality of off-times where the pulse signal is off, the on- and off-times being alternatingly arranged.

### CROSS-REFERENCES TO RELATED APPLICATIONS

This application is a continuation-in-part of U.S. non-provisional patent application Ser. No. 17/178,900 filed Feb. 18, 2021, which claims the priority of U.S. provisional patent application No. 62/979,772 filed Feb. 21, 2020.

### BACKGROUND OF THE INVENTION

### 1. Field of the Invention

The present invention relates to a method for controlling an electrochemical pump.

### 2. Description of the Prior Art

Injection, such as hypodermic injection or intravenous injection, is a common-seen method to deliver medicine into bodies. At present, pen-type injectors and electronically controlled injectors, such as patch-type injectors, wearable injectors and implanted injectors, have been developed to enable users to inject medicine into their own bodies by themselves. The conventional pen-type injectors utilize springs to generate driving force to deliver medicine which may cause sharp pain during injection, therefore, the pen-type injector can only inject small amount of medicine due to the pain, and cannot be used for injection of a large amount of medicament.

One electronically controlled injector delivers medicine via the driving force provided by a motor. The injection time and injection dosage can be controlled via controlling motor rotation. However, the electronically controlled injector with the motor is difficult to be miniaturized and is inconvenient for the patient for long-term carrying/wearing. Another conventional electronically controlled injector is only suitable to deliver insulin. For other macromolecular drugs (or biologics), such as monoclonal antibody, hormone, growth factor, and etc., the existing electronically controlled injectors are challenging of providing sufficient driving forces, especially in the case of delivering drugs in pre-filled containers (e.g., pre-filled syringe or pre-filled cartridge) due to the airtight seal between the rubber plunger and the glass of these containers. Further, to extend the lifetime of the electronically controlled injector devices, power administration is also critical and requires cutting-edge solutions.

Accordingly, it is highly desirable to provide a new pump technology capable of overcoming the abovementioned problems.

### SUMMARY OF THE INVENTION

The present invention provides a method for controlling an electrochemical pump. The method comprises the following steps: (i) providing an electrochemical pump comprising a substrate having an electrode region, a plurality of electrodes disposed in the electrode region, a dam enclosing the electrode region to define an accommodating space, an electrochemical liquid disposed in the accommodating space, and a control circuit electrically connected with the electrodes; and (ii) causing the control circuit to generate a pulse signal to enable an electrochemical reaction on surfaces of the electrodes. The pulse signal has alternating on-periods and off-periods where the pulse signal is off. In addition, each on-period has or is composed of a plurality of on-times where the pulse signal is on, and a plurality of off-times where the pulse signal is off, wherein the on- and off-times are alternatingly arranged.

Also provided is an electrochemical pump comprising: a substrate having an electrode region; a plurality of electrodes disposed in the electrode region; a dam enclosing the electrode region to define an accommodating space, the accommodating space storing an electrochemical liquid; a control circuit electrically connected to the electrodes; and a non-transitory machine-readable storage medium connected to the control circuit, including instructions that, when executed by the control circuit, causes the control circuit to: generate a pulse signal to enable an electrochemical reaction on surfaces of the electrodes, wherein the pulse signal has alternating on-periods and off-periods where the pulse signal is off, and wherein each on-period has a plurality of on-times where the pulse signal is on and a plurality of off-times where the pulse signal is off, the on- and off-times being alternatingly arranged.

Preferably, the pulse signal in each on-periods has a same amplitude, a same frequency, and a same duty cycle.

The on-times may have a frequency ranging from about 1 Hz to about 1 GHz.

According to certain embodiments of the present invention, the on-times have a frequency ranging from about 1 Hz to about 1 kHz. In such embodiments, the on- and off-times may be configured to have a duty cycle of about 50%, 55%, 60%, 65%, 70%, 75%, or more.

According to certain embodiments of the present invention, the on-times have a frequency ranging from about 1 kHz to about 1 MHz. In such embodiments, the on- and off-times may be configured to have a duty cycle of about 40%, 45%, 50%, 55%, 60%, 65%, 70%, 75%, or more.

According to certain embodiments of the present invention, the on-times have a frequency ranging from about 1 MHz to about 1 GHz. In such embodiments, the on- and off-times may be configured to have a duty cycle of about 25%, 30%, 35%, 40%, 45%, 50%, 55%, 60%, 65%, 70%, or 75%.

It was unexpectedly found in the present invention that a pulse signal of the present invention can achieve a delivery efficiency for an electrochemical pump similar to that resulted from a conventional pulse signal which is all “on” during its on-periods. As such, a method for controlling an electrochemical pump of the present invention has an advantage of power-saving.

The present invention also provides an electrochemical pump and a delivery device thereof, wherein a hybrid pulse is used to control the electrochemical reaction, whereby power is effectively saved and the lifetime of the electrochemical pump is significantly prolonged.

The present invention provides another electrochemical pump and a delivery device thereof, wherein edges of electrodes are covered with an insulating layer to protect the bonding between the electrodes and a substrate to allow high-power electrochemical reaction for high flow rate and/or large driving force.

In one embodiment, the electrochemical pump of the present invention comprises a substrate, a plurality of electrodes, a dam, and a control circuit. The substrate has an electrode region. The electrodes are disposed in the electrode region. The dam encircles the electrode region and defines an accommodating space. The accommodating space stores an electrochemical liquid. The control circuit is electrically connected with the electrodes and uses a pulse signal to selectively activate an electrochemical reaction on surfaces of the electrodes, wherein an enabling pulse of the pulse signal includes a plurality of sub-enabling pulses.

In one embodiment, the delivery device of the present invention comprises an electrochemical pump, a container, and a delivery connector. The electrochemical pump comprises a substrate, a plurality of electrodes, a dam, and a control circuit. The substrate has an electrode region. The electrodes are disposed in the electrode region. The dam encircles the electrode region and defines an accommodating space. The accommodating space stores an electrochemical liquid. The control circuit is electrically connected with the electrodes and uses a pulse signal to selectively activate an electrochemical reaction on surfaces of the electrodes, wherein an enabling pulse of the pulse signal includes a plurality of sub-enabling pulses. The container includes a sealing element and a piston. A liquid, which is to be delivered, is stored between the sealing element and the piston. The container is connected with the electrochemical pump, whereby an airtight room is defined between the piston and the accommodating space of the electrochemical pump. The delivery connector includes a tube, a puncture element and a delivery element. The puncture element is connected with one end of the tube and used to puncture the sealing element of the container, whereby the container is interconnected with exterior through puncture element. The delivery element is connected with another end of the tube and disposed on an object. The delivered liquid is pushed by the piston to arrive the object through the puncture element, the tube and the delivery element.

In another embodiment, the electrochemical pump of the present invention comprises a substrate, a plurality of electrodes, an insulating layer, a dam, and a control circuit. The substrate has an electrode region. The electrodes are disposed in the electrode region. The insulating layer covers edges of the electrodes and a portion of the electrodes is exposed. The dam encircles the electrode region and defines an accommodating space. The accommodating space stores an electrochemical liquid. The control circuit is electrically connected with the electrodes, selectively activating an electrochemical reaction on surfaces of the electrodes.

### BRIEF DESCRIPTION OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawings:

FIG. 1 is a schematic diagram of a delivery device according to a first embodiment of the present invention.

FIG. 2 is a schematic diagram of an electrochemical pump of a delivery device according to a second embodiment of the present invention.

FIG. 3 is a schematic diagram of an electrochemical pump of a delivery device according to a third embodiment of the present invention.

FIG. 4 is a schematic diagram of a substrate and electrodes of a delivery device according to a fourth embodiment of the present invention.

FIG. 5 is a schematic diagram of a pulse signal of a delivery device according to one embodiment of the present invention.

FIG. 6 is a schematic diagram of a delivery device according to a fifth embodiment of the present invention.

FIG. 7A and FIG. 7B show a comparison of delivery volume between a conventional pulse signal (“PWM1”) and a pulse signal of the present invention (“PWM2”).

### DESCRIPTION OF THE PREFERRED EMBODIMENTS

The following description is merely intended to illustrate various embodiments of the invention. As such, specific embodiments or modifications discussed herein are not to be construed as limitations to the scope of the invention. It will be apparent to one skilled in the art that various changes or equivalents may be made without departing from the scope of the invention.

In order to provide a clear and ready understanding of the present invention, certain terms are first defined. Additional definitions are set forth throughout the detailed description. Unless defined otherwise, all technical and scientific terms used herein have the same meanings as is commonly understood by one of skill in the art to which this invention belongs.

As used herein, the singular forms “a”, “an”, and “the” include plural referents unless the context clearly dictates otherwise. Thus, for example, reference to “a component” includes a plurality of such components and equivalents thereof known to those skilled in the art.

As used herein, the term “comprise” or “comprising” is generally used in the sense of include/including which means permitting the presence of one or more features, ingredients or components. The term “comprise” or “comprising” encompasses the term “consists” or “consisting of.”

In one aspect, the present invention provides a method for controlling an electrochemical pump. The method comprises providing an electrochemical pump and causing a control circuit of the electrochemical pump to generate a pulse signal to enable an electrochemical reaction. The electrochemical pump comprises a substrate having an electrode region, a plurality of electrodes disposed in the electrode region, a dam enclosing the electrode region to define an accommodating space, an electrochemical liquid disposed in the accommodating space, and a control circuit electrically connected with the electrodes.

In another aspect, the present invention provides an electrochemical pump comprising:
a substrate having an electrode region;
a plurality of electrodes disposed in the electrode region;
a dam enclosing the electrode region to define an accommodating space, the accommodating space storing an electrochemical liquid;
a control circuit electrically connected to the electrodes; and a non-transitory machine-readable storage medium connected to the control circuit, including instructions that, when executed by the control circuit, causes the control circuit to:
   generate a pulse signal to enable an electrochemical reaction on surfaces of the electrodes,
   wherein the pulse signal has alternating on-periods and off-periods where the pulse signal is off, and
   wherein each on-period has a plurality of on-times where the pulse signal is on and a plurality of off-times where the pulse signal is off, the on- and off-times being alternatingly arranged.

The pulse signal applied to the electrodes by the control circuit has alternating on-periods and off-periods where the pulse signal is off. In addition, each on-period has or is composed of a plurality of on-times where the pulse signal is on, and a plurality of off-times where the pulse signal is off, wherein the on- and off-times are alternatingly arranged.

According to the present invention, the on-times may have a frequency ranging from about 1 Hz to about 1 GHz.

In some embodiments, the on-times have a frequency ranging from about 1 Hz to about 1 kHz. In such embodiments, the on- and off-times may be configured to have a duty cycle of about 50%, 55%, 60%, 65%, 70%, 75%, 80%, 85%, 90%, 95%, or more, and up to about 99.99%.

In some other embodiments, the on-times have a frequency ranging from about 1 kHz to about 1 MHz. In such embodiments, the on- and off-times may be configured to have a duty cycle of about 40%, 45%, 50%, 55%, 60%, 65%, 70%, 75%, 80%, 85%, or 90%.

In some embodiments, the on-times have a frequency ranging from about 1 MHz to about 1 GHz. In such embodiments, the on- and off-times may be configured to have a duty cycle of about 25%, 30%, 35%, 40%, 45%, 50%, 55%, 60%, 65%, 70%, or 75%.

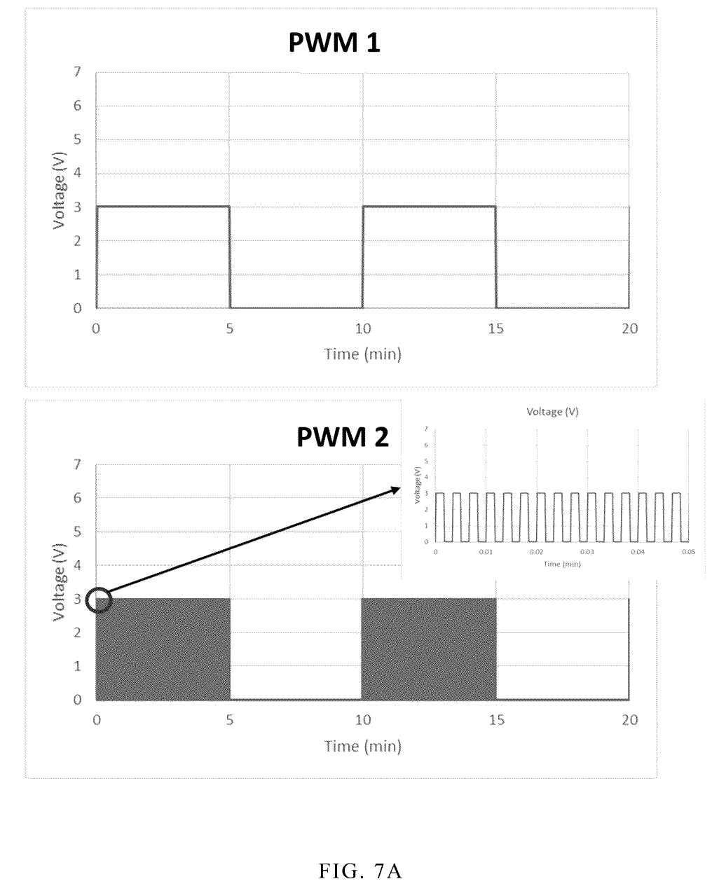

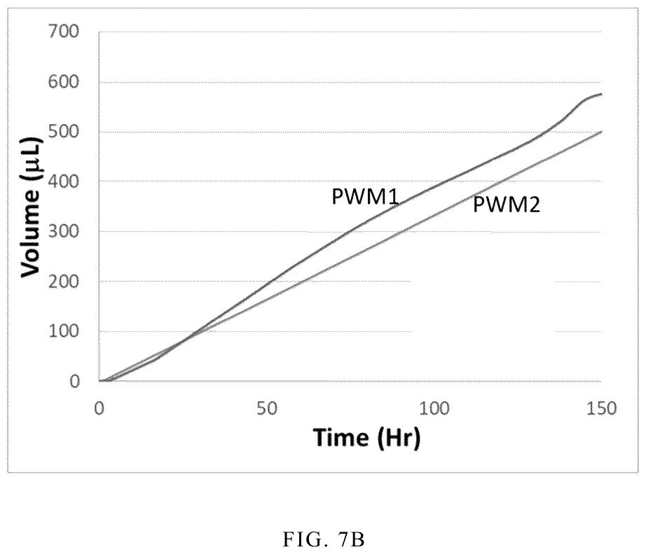

Conventionally, a pulse signal for enabling an electrochemical pump may have a typical frequency of about 0.00167 Hz (1/10/60 Hz) and a typical duty cycle of about 50% (e.g., PWM1 in FIG. 7A). By contrast, a waveform of a pulse signal of the present invention may be obtained by adding a plurality of periodical off-times in each on-periods of a conventional pulse signal to result in clusters of pulses (periodically generated “sub-pulse signals”) having a frequency within each on-periods much higher than that of the conventional pulse signal (e.g., 5 Hz for PWM2 in FIG. 7A).

According to certain preferred embodiments of the present invention, the pulse signal in each on-periods has a same amplitude, a same frequency, and a same duty cycle (more specifically, each sub-pulse signals may have a same duration of on-times and a same duration of off-times. In other words, the pulse signal in each on-periods may have a substantially same waveform.

As shown in FIGS. 7A and 7B, a pulse signal of the present invention PWM2 demonstrates a delivery efficiency for an electrochemical pump (volume of delivered liquid) similar to that of a conventional pulse signal PWM1. However, PWM2 uses substantially less power than PWM1 (about 50% less power, in view that the duty cycle for PWM2 during an on-period is about 50%). In addition, for pulses in an on-period set at a higher frequency, a duty cycle of less than 50% (e.g., about 45%, 40%, 35%, 30%, or as low as about 25%) may be used, while maintaining a delivery efficiency similar to that of a conventional counterpart.

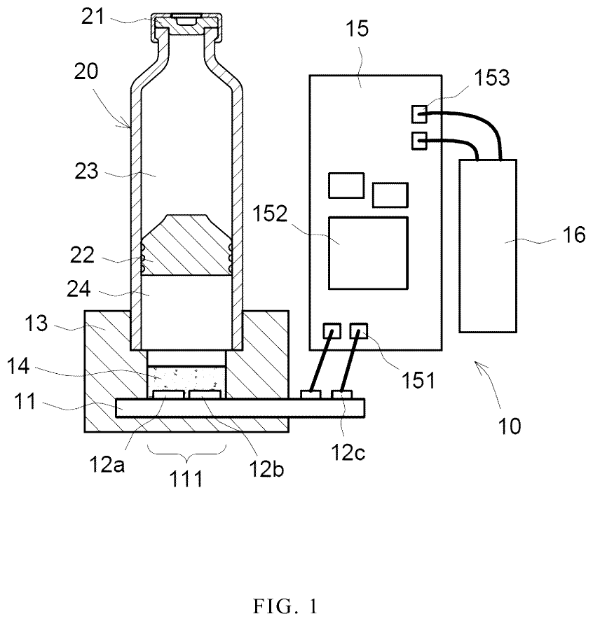

Please refer to FIG. 1. In one embodiment, the delivery device of the present invention comprises an electrochemical pump 10, a container 20 and a delivery connector 30. The container 20 includes a sealing element 21 and a piston 22. A storage space 23 is formed between the sealing element 21 and the piston 22 for storing a liquid, which is to be delivered. For example, the liquid to be delivered may be a drug or biologics such as monoclonal antibody. However, the liquid is not limited to be water-based fluid. The liquid may be a solvent-based fluid (such as DMSO) or an oil-based fluid (such as corn oil). In one embodiment, the container 20 may be a pre-filled container (prefilled syringe or prefilled cartridge).

The delivery connector 30 includes a tube 31, a puncture element 32 and a delivery element 33. The puncture element 32 is connected to one end of the tube 31 and used to puncture the sealing element 21 of the container 20, whereby the storage space 23 of the container 20 is interconnected with the exterior of the container 20 through puncture element 32. The delivery element 33 is connected to another end of the tube 31 and is to be disposed on an object. For example, the delivery element 33 may be inserted or implanted hypodermically, subcutaneously, intramuscularly, intravenously, or intraperitoneally. However, the delivery element 33 is not limited to be disposed in the abovementioned regions but may also be disposed on another appropriate region. The delivery element 33 shown in FIG. 1 is a needle-like structure. However, the delivery element 33 is not limited to be a needle-like structure but may be a connector, which is to be connected with another syringe or delivery instrument. Thereby, the delivery device of the present invention may be remote from the position where the drug is to be delivered. For example, the delivery device may be worn on the arm, abdomen, thigh or hips of a patient, and the needle is inserted into the adjacent area of the aforementioned position of the patient.

According to the abovementioned structure, after the puncture element 32 of the delivery connector 30 punctures the sealing element 21 of the container 20, the liquid (such as a drug), which is stored inside the storage space 23 for delivery, may be delivered through the puncture element 32, the tube 31 and the delivery element 33 to the object via pushing the piston 22.

The structure of the electrochemical pump 10 will be described in detail below. The electrochemical pump 10 of the present invention comprises a substrate 11, a plurality of electrodes 12a and 12b, a dam 13, and a control circuit 15. The substrate 11 has an electrode region 111, and the electrodes 12a and 12b is disposed in the electrode region 111 of the substrate 11. In one embodiment, the substrate 11 is made of glass, quartz, ceramic, semiconductor material or plastic. For example, the ceramic may be aluminum oxide or titanium oxide etc.; the semiconductor material may be silicon. The dam 13 encircles the electrode region 111 of the substrate 11 and defines an accommodating space for storing an electrochemical liquid 14. The control circuit 15 is electrically connected with the electrodes 12a and 12b. For example, the substrate 11 includes a plurality of electric-conduction contacts 12c, and the control circuit 15 includes electric-conduction contacts 151. Via leads or another appropriate means (such as connector or pogo pin), the electric-conduction contacts 151 of the control circuit 15 are electrically connected with the plurality of electric-conduction contacts 12c. Thereby, the control circuit 15 is electrically connected with the electrodes 12a and 12b. The control circuit 15 includes necessary electronic elements 152 (such as a microcontroller and passive elements) and electric-conduction contacts 153 for electric conduction with a power supply 16 (such as a battery). Neither the detailed structure of the control circuit 15 nor the connection means of the power supply 16 is the primary technical characteristic of the present invention. Therefore, they will not repeat herein.

The container 20 is connected with the electrochemical pump 10, and an airtight room 24 is defined between the piston 22 of the container 20 and the accommodating space formed by the dam 13. For example, an engagement structure corresponding to the container 20 is formed in the dam 13; while the container 20 is disposed into the engagement structure of the dam 13, the container 20 and the dam 13 define an airtight room 24 between the piston 22 and the electrochemical liquid 14. The control circuit 15 selectively supplies power to the electrodes 12a and 12b to selectively enable an electrochemical reaction on the surfaces of the electrodes 12a and 12b and generate gas. This additional gas increases the pressure inside the airtight room 24 and thus pushes the piston 22 to move.

In the embodiment shown in FIG. 1, the dam 13 contacts the outer wall of the container 20 to form the airtight room 24. However, the present invention is not limited by this embodiment. Please refer to FIG. 2. In one embodiment, the dam 13 contacts the inner wall of the container 20 to form the airtight room 24. In the embodiment shown in FIG. 2, the airtight room 24 interconnects with the accommodating space formed by the dam 13 through a passage 131. Thereby, the gas generated by the electrochemical reaction enters the airtight room 24 through the passage 131 to increase the pressure inside the airtight room 24.

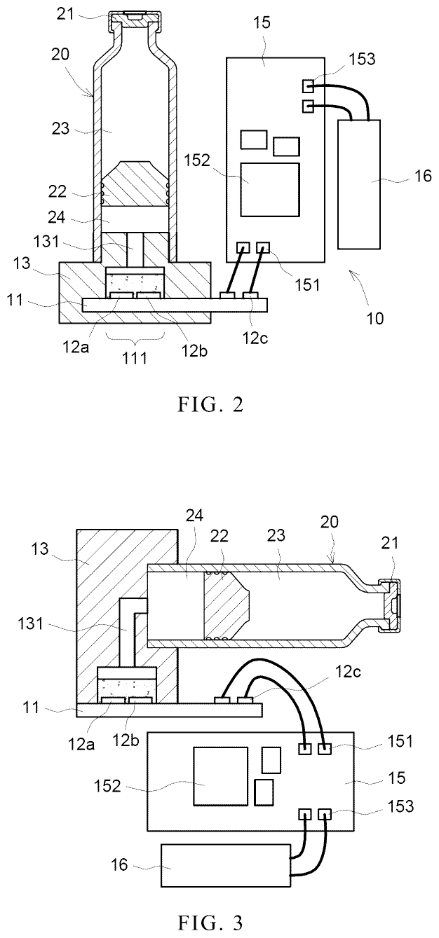

It would be appreciated that the design that the passage 131 is used to interconnect the airtight room 24 and the accommodating space formed by the dam 13 facilitates different designs of the relative position of the container 20 and the substrate 11. In the embodiments shown in FIG. 1 and FIG. 2, the container 20 is vertical to the substrate 11. However, the present invention is not limited to these embodiments. Please refer to FIG. 3. In one embodiment, the container 20 is parallel to the substrate 11. It should be noted: the present invention is not limited by the embodiments that the container 20 and the electrochemical pump 10 are directly connected to each other. In other embodiments, appropriate adapters may be used to connect the container 20 and the dam 13, whereby different containers or layouts may be used.

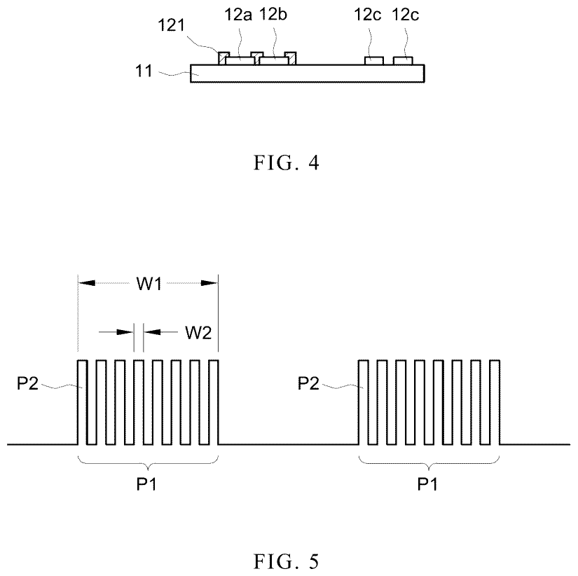

Please refer to FIG. 4. In one embodiment, the electrochemical pump 10 comprises an insulating layer 121, which covers the edges of the electrodes 12a and 12b and reveals a portion of the electrodes 12a and 12b, whereby to prevent from delamination of the electrodes. According to the abovementioned structure, the insulating layer 121 may increase the bonding strength between the substrate 11 and the electrodes 12a and 12b, decrease the chance that gas enters the interfaces between the substrate 11 and the electrodes 12a and 12b, and thus prevent from electrode delamination in a high-power electrochemical reaction. In one embodiment, the insulating layer 121 is made of epoxy (such as solder mask, SU-8), photo patternable polymer, photo patternable silicone, glass, ceramic, or plastic. For example, the photo patternable polymer includes photo resist, photo patternable polyimide, and photo patternable adhesives. In one embodiment, the insulating layer 121 can be formed by screen printing, semiconductor manufacturing, or sintering.

As mentioned above, the control circuit 15 selectively supplies power to enable an electrochemical reaction and generate gas on the surfaces of the electrodes 12a and 12b. Please refer to FIG. 5. In one embodiment, the control circuit 15 uses the pulse signal shown in FIG. 5 to selectively enable an electrochemical reaction on the surfaces of the electrodes 12a and 12b. The pulse signal shown in FIG. 5 includes two enabling pulses P1. It would be appreciated that the width W1 of the enabling pulse P1 may be modified according to a target delivery output of the electrochemical pump 10. For example, while the width W1 of the enabling pulse P1 is larger, the triggered electrochemical reaction is longer, and more gas is generated. Contrarily, while the width W1 of the enabling pulse P1 is smaller, the triggered electrochemical reaction is shorter, and less gas is generated. It would be appreciated that because the response of the electrochemical reaction is slower in comparison with the change of electric signal, gas is still generated between two enabling pulses P1. Therefore, appropriately adjusting the width W1 of the enabling pulse P1 may generate a required amount of gas and save energy. In one embodiment, the pulse signal according to the present invention can be realized by pulse width modulation (PWM) technology. It would be appreciated that setting the widths W1 of the enabling pulses P1 to be the same can also generate a predetermined amount of gas.

Particularly, in one embodiment, the enabling pulse P1 includes a plurality of sub-enabling pulses P2. The enabling pulse P1 is primary pulse width modulation and the sub-enabling pulse P2 is for a secondary pulse width modulation. Preferably, a width of the enabling pulse is 1/600 Hz, and a duty cycle of the enabling pulse is 50%, and a width of the sub-enabling pulse is 5 Hz, and a duty cycle of the sub-enabling pulse is 50%. In other words, while the enabling pulse P1 enables the electrochemical reaction, it does not activate the electrochemical reaction continuously but triggers the electrochemical reaction intermittently. Similar to that mentioned above, gas is still generated between two sub-enabling pulses P2. Therefore, the enabling pulse P1 formed by a plurality of sub-enabling pulses P2 may save energy furthermore. In one embodiment, the width W2 of the sub-enabling pulses P2 may be the same. It would be appreciated that the width W2 of the sub-enabling pulses P2 may be modified to adjust the target output of the electrochemical pump 10.

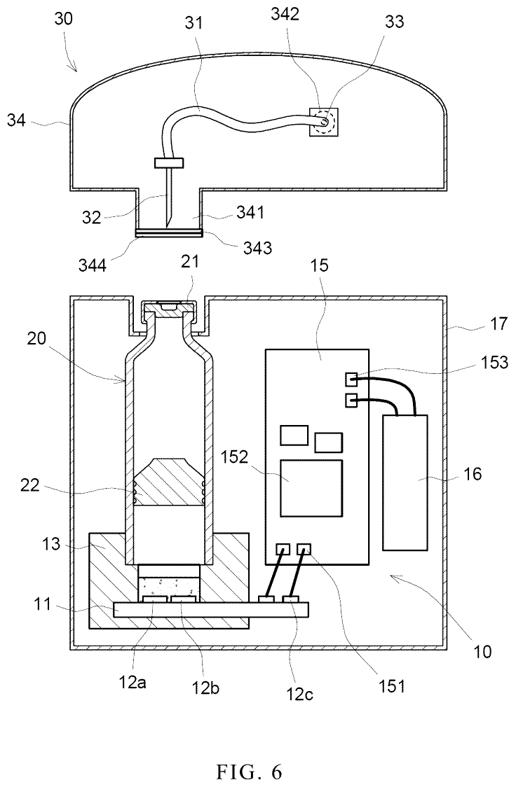

As mentioned above, one of the applications of the delivery device of the present invention is to deliver medicine to an object. Therefore, how to guarantee sterilization of the delivery path between the container 20 and the object is an important subject. Please refer to FIG. 6 for the solution of the abovementioned problem. In one embodiment, the delivery connector 30 further comprises a casing 34, and the tube 31, the puncture element 32 and the delivery element 33 are arranged in the interior of the casing 34. The casing 34 includes a first opening 341 and a second opening 342, wherein the puncture element 32 is corresponding to the first opening 341 and the delivery element 33 is corresponding to the second opening 342. The first opening 341 and the second opening 342 are respectively sealed by sealing membranes 343. Then, the delivery connector 30 having the abovementioned structure is sterilized. Thus, the interior of the casing 34 is maintained in a sterilized state. In other words, the tube 31, the puncture element 32 and the delivery element 33 are all in a sterilized state. In this embodiment, the electrochemical pump 10 and the container 20 are disposed inside a housing 17. While the present invention is to be used, the delivery connector 30 and a housing 17 are correspondingly assembled together. Thus, the puncture element 32 punctures the sealing membrane 343 on the first opening 341 and the sealing element 21 of the container 20. Similarly, the delivery element 33 punctures the sealing membrane on the second opening 342 and then is implanted into an appropriate position of the object. Thereby, the drug delivery path between the container 20 and the object is maintained in a sterilized state. In one embodiment, the sealing membrane on the first opening 341 and on the second opening 342 can be removed before the puncture element 32 is to puncture the sealing element 21 and the delivery element 33 is implanted into the object.

According to the abovementioned structure, it should be noted that the delivery connector 30, the container 20 and the electrochemical pump 10 may be fabricated by different manufacturers respectively and then assembled together to form a complete product. Thus, the high temperature used to sterilize the delivery connector 30 would not affect the stability of the drug in the container 20. Further, the demand to the cleanness of the environment where the parts are assembled together is lowered.

It would be appreciated that the outer surface of the sealing membrane 343 will be polluted after sterilization because it may contact the external environment. Therefore, a sterilizing process, such as swabbing the outer surface of the sealing membrane 343, may be used to decrease the risk of the pollution of the drug delivery path. Refer to FIG. 6 for an embodiment that can simplify the sterilization operation. In FIG. 6, the delivery connector 30 further comprises a protection layer 344, which is disposed on the outer surface of the sealing membrane 343. According to the abovementioned structure, while the present invention is to be used, only removing the protection layer 344 is sufficient to guarantee the sterilized state of the sealing membrane 343. Therefore, the protection layer 344 can secure the sterilization of the sealing membrane 343 and simplify the operation process of using the present invention.

In conclusion, the electrochemical pump and the delivery device of the present invention use hybrid pulses to control an electrochemical reaction, whereby electric energy is effectively saved and the usage time of the electrochemical pump is significantly prolonged. Further, an electrochemical pump and a delivery device of the present invention includes an insulating layer covering the edges of the electrodes to enhance the bonding strength between the electrodes and the substrate and decrease the chance that gas enters the interfaces between the electrodes and the substrate, whereby to prevent from electrode delamination.

While preferred embodiments of the present invention have been shown and described herein, it will be obvious to those skilled in the art that such embodiments are provided by way of example only and can be implemented in combinations. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for controlling an electrochemical pump, the method comprising the steps of:
providing an electrochemical pump comprising: a substrate having an electrode region; a plurality of electrodes disposed in the electrode region; a dam enclosing the electrode region to define an accommodating space; an electrochemical liquid disposed in the accommodating space; and a control circuit electrically connected with the electrodes; and
causing the control circuit to generate a pulse signal to enable an electrochemical reaction on surfaces of the electrodes;
wherein the pulse signal has alternating on-periods and off-periods where the pulse signal is off, and
wherein each on-periods has a plurality of on-times where the pulse signal is on and a plurality of off-times where the pulse signal is off, the on- and off-times being alternatingly arranged.

2. The method of claim 1, wherein the pulse signal in each on-periods has a same amplitude, a same frequency, and a same duty cycle.

3. The method of claim 1, wherein the on-times have a frequency ranging from about 1 Hz to about 1 GHz.

4. The method of claim 3, wherein the on-times have a frequency ranging from about 1 Hz to about 1 kHz.

5. The method of claim 4, wherein the on- and off-times have a duty cycle of about 50%, 55%, 60%, 65%, 70%, or 75%.

6. The method of claim 3, wherein the on-times have a frequency ranging from about 1 kHz to about 1 MHz.

7. The method of claim 6, wherein the on- and off-times have a duty cycle of about 40%, 45%, 50%, 55%, 60%, 65%, 70%, or 75%.

8. The method of claim 3, wherein the on-times have a frequency ranging from about 1 MHz to about 1 GHz.

9. The method of claim 8, wherein the on- and off-times have a duty cycle of about 25%, 30%, 35%, 40%, 45%, 50%, 55%, 60%, 65%, 70%, or 75%.

10. An electrochemical pump comprising:
a substrate having an electrode region;
a plurality of electrodes disposed in the electrode region;
a dam enclosing the electrode region to define an accommodating space, the accommodating space storing an electrochemical liquid;
a control circuit electrically connected to the electrodes; and
a non-transitory machine-readable storage medium connected to the control circuit, including instructions that, when executed by the control circuit, causes the control circuit to:
generate a pulse signal to enable an electrochemical reaction on surfaces of the electrodes,
wherein the pulse signal has alternating on-periods and off-periods where the pulse signal is off, and
wherein each on-period has a plurality of on-times where the pulse signal is on and a plurality of off-times where the pulse signal is off, the on- and off-times being alternatingly arranged.

11. The electrochemical pump of claim 10, wherein the pulse signal in each on-periods has a same amplitude, a same frequency, and a same duty cycle.

12. The electrochemical pump of claim 10, wherein the on-times have a frequency ranging from about 1 Hz to about 1 GHz.

13. The electrochemical pump of claim 12, wherein the on-times have a frequency ranging from about 1 Hz to about 1 kHz.

14. The method of claim 13, wherein the on- and off-times have a duty cycle of about 50%, 55%, 60%, 65%, 70%, or 75%.

15. The method of claim 12, wherein the on-times have a frequency ranging from about 1 kHz to about 1 MHz.

16. The method of claim 15, wherein the on- and off-times have a duty cycle of about 40%, 45%, 50%, 55%, 60%, 65%, 70%, or 75%.

17. The method of claim 12, wherein the on-times have a frequency ranging from about 1 MHz to about 1 GHz.

18. The method of claim 17, wherein the on- and off-times have a duty cycle of about 25%, 30%, 35%, 40%, 45%, 50%, 55%, 60%, 65%, 70%, or 75%.
